(19) United States
(12) Patent Application Publication
(10) Pub. No.: US 2019/0204335 A1
(43) Pub. Date: Jul. 4, 2019

(54) Assay Method For Determining The Risk Of Pre-Eclampsia

(71) Applicant: The Queen's University of Belfast, Belfast (IE)

(72) Inventors: Lana MCCLEMENTS, Belfast (IE); Tracy ROBSON, Belfast (IE); Timothy LYONS, Charleston, SC (US)

(21) Appl. No.: 16/333,889

(22) PCT Filed: Sep. 15, 2017
(86) PCT No.: PCT/GB2017/052751

§ 371 Date: Mar. 15, 2019

(30) Foreign Application Priority Data
Sep. 15, 2016 (GB) 1615753.9

(51) Int. Cl. G01N 33/68

(52) U.S. Cl. CPC G01N 33/689; G01N 2800/368

(57) ABSTRACT

The present invention relates to the detection of FK506-binding protein like (FKBPL) in samples from pregnant women and the use of FKBPL as a predictive biomarker for the development of pre-eclampsia. The present invention further relates to kitsfor the detection of FKBPL and methods of preventing pre-eclampsia.

### FIELD OF THE INVENTION

The present invention relates to the detection of FK506-binding protein like (FKBPL) in samples from pregnant women and the use of FKBPL as a predictive biomarker for the development of pre-eclampsia. The present invention further relates to kits for the detection of FKBPL and methods of preventing pre-eclampsia.

### BACKGROUND

Pre-eclampsia (PE) is a condition that affects some pregnant women, with symptoms appearing usually after 34 weeks gestation. It occurs in 5-8% of pregnancies causing significant maternal and fetal morbidity and mortality.

At present, PE is diagnosed based on clinical symptoms, for example, elevated blood pressure with or without proteinuria. In the absence of proteinuria, any of the following symptoms such as thrombocytopenia (platelets<100,000 μl), impaired liver function, progressive renal insufficiency, pulmonary oedema or cerebral or visual disturbances, will also indicate the diagnosis of PE. However, clinical criteria alone may be inadequate to predict adverse outcomes.

There is therefore a need for a biochemical assay to predict the risk of PE developing early in pregnancy. This would allow for closer monitoring of women who are at high risk of developing PE to prevent complications associated with underlying PE, and would allow for potential new treatments to be started early in pregnancy. Such a test may also reduce the costly emergency care required for women diagnosed with PE late in pregnancy or who are not accurately diagnosed and premature births.

### SUMMARY OF THE INVENTION

The present inventors have determined that FKBPL protein and/or gene expression levels can provide an early indication if a pregnant woman is at increased risk of PE.

This is based on their determination that FKBPL protein expression levels decrease during pregnancy in women who develop PE.

Accordingly, the present invention provides a method of determining the risk of pre-eclampsia in a pregnant woman, the method comprising:
a) quantifying an expression level of FKBPL in a biological sample taken from the pregnant woman at a first time point;
b) quantifying the expression level of FKBPL in a biological sample taken from the pregnant woman at a second time point, wherein the second time point is later in the pregnancy than the first time point;
c) comparing the expression levels at the first time point and the second time point;

wherein if there is a decrease in the expression level from the first time point to the second time point, the woman is at increased risk of developing pre-eclampsia.

The present invention also provides a method of determining the risk of pre-eclampsia in a pregnant woman, the method comprising:
a) taking a first biological sample from the pregnant woman at a first time point;
b) quantifying an expression level of FKBPL in the first biological sample;
c) taking a second biological sample from the pregnant woman at a second time point, wherein the second time point is later in the pregnancy than the first time point;
d) quantifying the expression level of FKBPL in the second biological sample; and
e) comparing the expression levels at the first time point and the second time point,
   wherein if there is a decrease in the expression level from the first time point to the second time point, the woman is at increased risk of developing pre-eclampsia. Optionally, the method further comprises selecting the woman for additional pre-natal monitoring if a decrease in the expression level from the first time point to the second time point is detected.

In a further aspect of the invention there is provided a method of detecting FKBPL in a pregnant woman, the method comprising:
a) taking a first biological sample from the pregnant woman at a first time point; and
b) quantifying the FKBPL in the first biological sample by contacting the sample with an anti-FKBPL antibody and detecting binding between FKBPL and the antibody. Preferably, this method may further comprise taking a second biological sample from the pregnant woman at a second time point and quantifying the FKBPL protein levels in the second biological sample by contacting the sample with an anti-FKBPL antibody and detecting binding between FKBPL and the antibody, wherein the second time point is later in the pregnancy than the first time point. Optionally, the method involves first selecting a pregnant woman for testing and/or comparing the FKBPL levels at the first time point and the second time point.

In a further aspect of the invention, there is provided a method of detecting FKBPL in a pregnant woman, the method comprising quantifying the FKBPL protein levels in a first biological sample taken from the pregnant woman at a first time point by contacting the first biological sample with an anti-FKBPL antibody and detecting binding between FKBPL and the antibody. Preferably, this method may further comprise quantifying the FKBPL protein levels in a second biological sample taken from the pregnant woman at a second time point by contacting the second biological sample with an anti-FKBPL antibody and detecting binding between FKBPL and the antibody, wherein the second time point is later in the pregnancy than the first time point. Optionally, the method involves first selecting a pregnant woman for testing and/or comparing the FKBPL levels at the first time point and the second time point.

In a further aspect of the invention, there is provided is a method of determining the risk of pre-eclampsia where only one biological sample is taken. For example,
quantifying an expression level of FKBPL in a biological sample taken from a pregnant woman at between weeks 5-30 weeks of pregnancy;
comparing the expression level of FKBPL to a control standard or the expression of FKBPL in a control sample;
   wherein if there is a decrease in the FKBPL expression in the biological sample compared with the control standard or the expression of FKBPL in the control sample, the woman is at increased risk of developing PE.

In a further aspect of the invention, provided is a method of preventing pre-eclampsia in a pregnant woman, the method comprising:
a) taking a first biological sample from the pregnant woman at a first time point;
b) quantifying an expression level of FKBPL in the first biological sample;
c) taking a second biological sample from the pregnant woman at a second time point, wherein the second time point is later in the pregnancy than the first time point;
d) quantifying the expression level of FKBPL in the second biological sample; and
e) comparing the expression levels at the first time point and the second time point, and
f) administering a therapeutically effective amount of a FKBPL agonist if there is a decrease in the expression level from the first time point to the second time point.

In a further aspect of the invention, provided is a FKBPL agonist for use in a method of preventing pre-eclampsia in a pregnant woman. Optionally, the method further comprises:
a) quantifying an expression level of FKBPL in a biological sample taken from the pregnant woman at a first time point;
b) quantifying the expression level of FKBPL in a biological sample taken from the pregnant woman at a second time point, wherein the second time point is later in the pregnancy than the first time point;
c) comparing the expression levels at the first time point and the second time point; and
d) administering a therapeutically effective amount of the FKBPL agonist if there is a decrease in the expression level from the first time point to the second time point.

The FKBPL agonist may be a statin, optionally pravastatin; a fibrate, optionally fenofibrate; a stilbenoid, optionally resveratrol; or metformin.

With reference to the time points for taking biological samples from the pregnant woman, the first time point may be taken between weeks 3-14 of pregnancy, preferably weeks 10-14 of pregnancy; and the second time point may be taken between weeks 15-30 of pregnancy, preferably between weeks 15-23 of pregnancy. Where only one sample is taken, this may be taken from weeks 10-20 of pregnancy.

The biological sample may be blood, plasma, serum or placenta. The FKBPL expression levels are preferably determined using a protein which specifically binds FKBPL protein, for example an antibody.

In a further aspect of the invention, provided is use of FKBPL as a biomarker for pre-eclampsia in pregnant women.

In a further aspect of the invention, provided is a kit comprising a reagent selected from: an antibody, antibody derivative or antibody fragment with binding specificity for FKBPL, for use in a method of diagnosing whether a pregnant women is at increased risk of pre-eclampsia.

Determining or testing the FKBPL expression levels may involve quantifying FKBPL protein expression or secretion; and/or FKBPL gene expression.

The pregnant woman to be tested may have pre-gestational diabetes mellitus.

These and further aspects of the invention are described by way of example only in further detail below and with reference to the accompanying examples.

### DESCRIPTION OF THE FIGURES

FIG. 1 shows upregulation of FKBPL protein levels in HTR8.SV.neo cells after treatment with highly oxidised and glycated lipoproteins (HOG-LDL; representing oxidative stress). Treatment was carried out using 25 μg/ml of HOG-LDL or native (N)-LDL for 24 hours, in non-serum containing medium. Error bars: SEM; n≥3 (unpaired t-test).

FIG. 2 shows upregulation of FKBPL mRNA levels following 48 h treatment with high glucose (10-40 mM) in BeWO cells (n=3, t=48 h). The change was measured as fold change in FKBPL mRNA compared to control glucose concentration (5.5 mM). Error bars: SEM; n≥3.

FIG. 3 shows downregulation of FKBPL protein expression in HTR8.SV.neo trophoblast cells following exposure to hypoxia for 24 h. Error bars: SEM;

FIG. 4 shows downregulation of FKBPL protein expression in HUVEC (human umbilical vein endothelial cells) following exposure to hypoxia for 24 h. Error bars: SEM; n≥3.

FIG. 5 shows downregulation in FKBPL mRNA levels in HTR8.SV.neo cells following treatment with DMOG (HIF-1α activator; 10 μM) for 24 h. No change was observed following treatment with 4-HNE (lipid peroxidation product; 10 μM) in serum-containing medium. Error bars: SEM, n≥3.

FIG. 6 shows downregulation of FKBPL protein expression following treatment with 4-HNE (10 μM) or DMOG (10 μM) in the BeWo trophoblast cell line in serum-containing medium. Error bars: SEM, n≥3.

FIG. 7 shows FKBPL protein expression within chorionic villi is detected strongly in syncytiotrophoblasts and endothelial cells (A). CD31 staining of endothelial/blood vessels in chorionic villi is also presented (B).

FIG. 8 shows downregulation in FKBPL plasma levels between first and second trimester only in women with type 1 diabetes who developed PE. Plasma FKBPL levels were measured using ELISA and Wilcoxon signed rank test performed between first and second trimester. DM-: non-diabetic (v1, n=16; v2, n=12; v3, n=12); DM+PE-: type 1 diabetic without preeclampsia (v1, n=19; v2, n=16; v3, n=13); DM+PE+: type 1 diabetic with preeclampsia (v1, n=17; v2, n=14; v3, n=16). Error bars: standard deviation.

FIG. 9 shows lower plasma FKBPL levels in nulliparous women without diabetes at 15 weeks gestation who developed pre-eclampsia (PE, n=52) compared to healthy controls (Control, n=53). Student t-test was performed to determine any significant differences between the group of women who developed pre-eclampsia and healthy controls. Error bars: standard deviation.

FIG. 10 shows that fenofibrate increases the FKBPL protein levels in non-diabetic rats at 5 weeks. Rats were made diabetic using streptozotocin (STZ) injection and fenofibrate treatment was given as part of the Chow food for 4 weeks. Error bars: SEM; n=6 in each group.

FIG. 11 shows that fenofibric acid (FFA; 10 μM) increases the FKBPL protein levels in HTR8.SV.neo trophoblast cells. Fenofibric acid (FFA) is an active form of fenofibrate in cell culture. Relative protein levels normalised to the vehicle control were determined. Error bars: SEM; n=3.

FIGS. 12a and 12b show metformin (10 μM) upregulates FKBPL (A) in HUVEC following exposure to hypoxia (1%). (B) Western blots of FKBPL, Sirtuin-1 (SIRT1) levels (control) and alpha-tubulin (a house keeping protein) are shown. FKBPL expression was normalised to vehicle control treatment. VC—vehicle control. Error bars: SEM, n≥3

FIGS. 13 shows that (A) metformin treatment (10 μM) does not affect levels of FKBPL in HUVEC in normoxia conditions. (B) Western blots of FKBPL, Sirtuin-1 (SIRT-1) levels (control) and alpha-tubulin (a house keeping protein) are shown. FKBPL expression was normalised to vehicle control treatment. Error bars: SEM, n≥3.

### DETAILED DESCRIPTION

Pre-eclampsia (PE) is a disorder that occurs only during pregnancy and affects both the mother and the unborn baby. Affecting at least 5-8% of all pregnancies, it is a rapidly progressive condition characterized by high blood pressure and the presence of protein in the urine. Ankle swelling, sudden weight gain, headaches and changes in vision are further important symptoms. However, some women with rapidly advancing disease report few symptoms.

Typically, PE occurs late in pregnancy: in the 3rd trimester. Proper pre-natal care is essential to diagnose and manage PE. Globally, PE and other hypertensive disorders of pregnancy are a leading cause of maternal and infant illness and death. By conservative estimates, these disorders are responsible for 76,000 maternal and 500,000 infant deaths each year. The exact cause of PE is unknown. However, experts believe it may be due to inappropriate placental development due to restricted blood supply of oxygen and nutrients.

### FKBPL as a Biomarker

The inventors have found that FKBPL can be used as a biomarker for the development of PE. That is, the level of FKBPL in a sample from a pregnant woman can give an indication as to the risk or likelihood of developing PE.

### FKBPL

FKBPL belongs to the family of FK506 binding proteins. It is an important regulator of angiogenesis and targets the CD44 pathway.

By “FKBPL”, it is meant to include full length FKBPL as well as FKBPL variants, FKBPL fragments and FKBPL variant fragments. For brevity, FKBPL is used to denote both the FKBPL protein and FKBPL gene.

FKBPL can be encoded by the nucleotide sequence:

(Accesion number NM_022110)
(SEQ ID NO 1)
atggagacgc caccagtcaa tacaattggagaaaaggaca
cctctcagcc gcaacaagag tgggaaaaga accttcggga
gaaccttgattcagttattc agattaggca gcagccccga
gaccctccta ccgaaacgct tgagctggaagtaagcccag
atccagccag ccaaattcta gagcatactc aaggagctga
aaaactggttgctgaacttg aaggagactc tcataagtct
catggatcaa ccagtcagat gccagaggcccttcaagctt
ctgatctctg gtactgcccc gatgggagct ttgtcaagaa
gatcgtaatccgtggccatg gcttggacaa acccaaacta
ggctcctgct gccgggtact ggctttggggtttcctttcg
gatcagggcc gccagagggc tggacagagc taactatggg
cgtagggccatggagggagg aaacttgggg ggagctcata
gagaaatgct tggagtccat gtgtcaaggtgaggaagcag
agcttcagct gcctgggcac tctggacctc ctgtcaggct
cacactggcatccttcactc aaggccgaga ctcctgggag
ctggagacta gcgagaagga agccctggccagggaagaac
gtgcaagggg cacagaacta tttcgagctg ggaaccctga
aggagctgcccgatgctatg gacgggctct tcggctgctc
ctgactttac ccccacctgg ccctccagaacgaactgtcc
ttcatgccaa tctggctgcc tgtcagttgt tgctagggca
gcctcagttggcagcccaga gctgtgaccg ggtgttggag
cgggagcctg gccatttaaa ggccttataccgaagggggg
ttgcccaggc tgcccttggg aacctggaaa aagcaactgc
tgacctcaagaaggtgctgg cgatagatcc caaaaaccgg
gcagcccagg aggaactggg gaaggtggtcattcagggga
agaaccagga tgcagggctg gctcagggtc tgcgcaagat
gtttggctgattaaaagtta aaccttaaaa gagaaaaaaa
aaaaaaa,

and can have the amino acid sequence:

(SEQ ID NO 2)
METPPVNTIGEKDTSQPQQEWEKNLRENLDSVIQIRQQPRDPPTETLELE
VSPDPASQILEHTQGAEKLVAELEGDSHKSHGSTSQMPEALQASDLWYCP
DGSFVKKIVIRGHGLDKPKLGSCCRVLALGFPFGSGPPEGWTELTMGVGP
WREETWGELIEKCLESMCQGEEAELQLPGHSGPPVRLTLASFTQGRDSWE
LETSEKEALAREERARGTELFRAGNPEGAARCYGRALRLLLTLPPPGPPE
RTVLHANLAACQLLLGQPQLAAQSCDRVLEREPGHLKALYRRGVAQAALG
NLEKATADLKKVLAIDPKNRAAQEELGKVVIQGKNQDAGLAQGLRKMFG
or be a variant or fragment thereof.

The FKBPL protein variant can have 25 or fewer, more preferably 15 or fewer, even more preferably of 10 or fewer, 2 or fewer amino acids inserted, deleted or substituted into FKBPL (SEQ ID NO 2), whilst providing a protein with FKBPL activity.

Alternatively, a variant may have at least 30%, 40%, 50%, 60%, 70%, 80%, 90%, 95% or 99% identity to FKBPL (SEQ ID No 2) whilst retaining FKBPL activity.

Various functional assays can be used to test for FKBPL activity. For example, FKBPL is an anti-angiogenic protein. The anti-angiogenic activity of FKBPL can be tested as described in Valentine et al., (2011): Clinical Cancer Research, Mar 1;17(5):1044-56 and Yakkundi et al., (2013): PLoS One;8(2):e55075. Briefly, the activity of FKBPL can be assessed in a range of functional cell assays. The ability of FKBPL to inhibit migration, and Matrigel-dependent tubule formation can be determined. Further tests in an ex vivo rat model of neovascularization and in in vivo mouse models of angiogenesis, that is, the sponge implantation and the intravital microscopy models, can be carried out. Antitumor efficacy can be determined in human tumor xenograft models.

Alternatively, a FKBPL variant may be encoded by a nucleic acid sequence comprising:

(SEQ ID No 3)
atggagacgc caccagtcaa tacaattggagaaaaggaca
cctctcagcc gcaacaagag tgggaaaaga accttcggga
gaaccttgattcagttattc agattaggca gcagccccga
gaccctccta ccgaaacgct tgagctggaagtaagcccag
atccagccag ccaaattcta gagcatactc aaggagctga
aaaactggttgctgaacttg aaggagactc tcataagtct
catggatcaa ccagtcagat gccagaggcccttcaagctt
ctgatctctg gtactgcccc gatgggagct ttgtcaagaa
gatcgtaatccgtggccatg gcttggacaa acccaaacta
ggctcctgct gccgggtact ggctttggggtttcctttcg
gatcagggcc gccagagggc tggacagagc taactatggg
cgtagggccatggagggagg aaacttgggg ggagctcata
gagaaatgct tggagtccat gtgtcaaggtgaggaagcag
agcttcagct gcctgggcac actggacctc ctgtcgggct
cacactggcatccttcactc aaggccgaga ctcctgggag
ctggagacta gcgagaagga agccctggccagggaagaac
gtgcaagggg cacagaacta tttcgagctg ggaaccctga
aggagctgcccgatgctatg gacgggctct tcggctgctc
ctgactttac ccccacctgg ccctccagaacgaactgtcc
ttcatgccaa tctggctgcc tgtcagttgt tgctagggca
gcctcagttggcagcccaga gctgtgaccg ggtgttggag
cgggagcctg gccatttaaa ggccttataccgaagggggg
ttgcccaggc tgcccttggg aacctggaaa aagcaactgc
tgacctcaagaaggtgctgg cgatagatcc caaaaaccgg
gcagcccagg aggaactggg gaaggtggtcattcagggga
agaaccagga tgcagggctg gctcagggtc tgcgcaagat
gtttggctgattaaaagtta aaccttaaaa gagaaaaaaa
aaaaaaa

and have the amino acid sequence:

(SEQ ID No 4)
METPPVNTIGEKDTSQPQQEWEKNLRENLDSVIQIRQQPRDPPTETLELE
VSPDPASQILEHTQGAEKLVAELEGDSHKSHGSTSQMPEALQASDLWYCP
DGSFVKKIVIRGHGLDKPKLGSCCRVLALGFPFGSGPPEGWTELTMGVGP
WREETWGELIEKCLESMCQGEEAELQLPGHTGPPVGLTLASFTQGRDSWE
LETSEKEALAREERARGTELFRAGNPEGAARCYGRALRLLLTLPPPGPPE
RTVLHANLAACQLLLGQPQLAAQSCDRVLEREPGHLKALYRRGVAQAALG
NLEKATADLKKVLAIDPKNRAAQEELGKVVIQGKNQDAGLAQGLRKMFG.

A fragment of FKBPL/FKBPL variant may comprise a stretch of amino acid residues of at least 5 to 7 contiguous amino acids, often at least 7 to 9 contiguous amino acids, typically 9 to 13 contiguous amino acids, more preferably at least 20 to 30 or more contiguous amino acids, most preferably at least 30 to 40 or more consecutive amino acids. The fragment may retain FKBPL activity, the test for which is described above.

For brevity, only FKBPL is referred to in the explanation of the invention which follows. However, the following explanation of the methods and kits of the invention equally applies to FKBPL variants, FKBPL fragments and FKBPL variant fragments.

### Detecting FKBPL

By detect is meant determining if an interaction between two agents for example two proteins or two nucleic acids is present or absent. For example, the interaction of

FKBPL with an antibody specific for FKBPL; or FKBPL mRNA with a nucleic acid probe specific for the FKBPL gene.

Detection may include quantification. Detection may include the use of an agent which is capable of detection (a label) using for example spectrophotometry, flow cytometry, or microscopy. Exemplary labels include radioactive isotopes (such as ³H, ¹⁴C, ¹⁵N, ³⁵S, ⁹⁰V, ⁹⁹Tc, ¹¹¹Ln, ¹²⁵I, or ¹³¹I), fluorophores (such as fluorescein, fluorescein isothiocyanate, rhodamine or the like), chromophores, ligands, chemiluminescent agents, bioluminescent agents (such as luciferase, green fluorescent protein (GFP) or yellow fluorescent protein), enzymes that can produce a detectable reaction product (such as horseradish peroxidise, luciferase, alkaline phosphatase, beta-galactosidase) and combinations thereof. Specific examples of detection are given below.

### Quantifying the Expression Level of FKBPL

Quantifying the expression level of FKBPL involves testing the biological samples taken from the pregnant woman for the amount of FKBPL in the sample.

This can involve testing for the secretion of FKBPL protein into the biological sample. For example, the secretion of FKBPL into the blood from: a) endothelial cells and/or b) placenta.

Alternatively or in addition, the expression of the FKBPL gene within the placental tissue can be tested.

### Quantification of Protein Expression and Secretion

Immunohistochemistry (IHC) can be used to detect protein expression. Western blotting and ELISA are methods useful for detecting protein expression and secretion. Antibodies which specifically bind FKBPL may be used in these methods. IHC samples can be analysed using an automated image analysis system, so as to provide a blinded analysis. For this, whole-slide digital images can be first captured at 20× using a ScanScope XT Slide Scanner (Aperio Technologies). Secondly, a positive pixel count algorithm (Aperio Technologies) can be used to develop a quantitative scoring model for FKBPL expression. Statistical analysis of tissue microarray-derived data can be carried out using the v2 test for trend, Fisher's exact and Mann-Whitney tests and Kaplan-Meier plots can be used for survival analysis and the curves compared using the log-rank test. Cox proportional hazards regression can be used to estimate proportional hazard ratios and conduct multivariate analyses as described previously. All calculations can be performed with SPSS v11.0 (SPSS, IL). In addition, to facilitate generation of discrete multi-marker test, fluorescently-tagged antibodies (carrying non-overlapping fluorophores) against FKBPL then additional relevant placental biomarkers can be used simultaneously. Advantageously a recently developed fluorescent scanning system from Aperio, for example, the ScanScope FL system could be used. This assay method would provide a further layer of sophistication by providing more quantitative analysis than that afforded by conventional brightfield imaging.

An Elisa sandwich enzyme immunoassay can be used, for example the ELISA Kit for FK506 Binding Protein Like Protein from Cloud-Clone Corp (Cat no. SEL523Hu). This kit provides a mictotiter plate pre-coated with an antibody specific to FKBPL. Standards or samples are then added to the plate wells followed by a biotin-conjugated antibody specific to FKBPL. Next, Avidin conjugated to Horseradish Peroxidase (HRP) is added to each microplate well and incubated. After TMB substrate solution (3,3′,5,5′-Tetramethylbenzidine: a chromogenic substrate which acts as a hydrogen donor for the reduction of hydrogen peroxide to water by HRP) is added, only those wells that contain FKBPL, biotin-conjugated antibody and enzyme-conjugated Avidin will exhibit a change in color. The enzyme-substrate reaction is terminated by the addition of sulphuric acid solution and the color change is measured spectrophotometrically at a wavelength of 450 nm±10 nm. The concentration of FKBPL in the samples is then determined by comparing the O.D. of the samples to a standard curve.

Western Blotting involves: 1) separating proteins by size by gel electrophoresis; 2) transfering the proteins from the gel to a membrane solid support, for example PVDF membrane; and 3) visualizing the target protein using a primary and optionally a secondary antibody. The bound primary or secondary antibody is detectable, for example, as a result of conjugation to HRP. The resulting signal after adding a chromogenic substrate allows quantification of the original protein band in the gel, for example, using ImageJ software (NIH, USA) and after adjusting to a standard used in the protocol. Optimally, the anti-FKBPL antibody is the FKBPL rabbit polyclonal primary antibody from ProteinTech IL, USA (Cat no. 10060-1-AP). To detect this primary antibody, an anti-rabbit IgG horseradish peroxidase secondary antibody (GE Healthcare, Cat no. NA934V) may be used.

Protein which Specifially Binds FKBPL

FKBPL protein can be detected using a primary antibody with binding specificity to FKBPL. The primary antibody can be labelled with a detectable moiety or can be conjugated to a hapten (such as biotin or the like) wherein the hapten is detectable by a detectably labelled cognate hapten binding molecule, for example streptavidin horseradish peroxidase. Alternatively, a secondary antibody can be used which specifically binds the first primary antibody and instead this secondary antibody may be detectable as described above for the primary antibody.

The binding specificity of FKBPL antibodies (antibodies with binding specificity to FKBPL) can be established using Western blotting, in parallel with immunohistochemical analysis of formalin-fixed, paraffin-embedded cell lines mimicking the placental samples. In brief, a placental cell line +/− FKBPL targeted siRNA, together with FKBPL overexpressing stable clones may be used to optimize anti-FKBPL antibodies. Cell lines may be fixed in PFA for 30 min and resuspended in 70% ethanol overnight before being embedded in paraffin and arrayed using a tissue arrayer. Immunocytochemically stained cell pellet arrays may then be compared with Western blot data to check the specificity and suitability of the antibodies and the significance of correlations determined using Spearman's rank test. The antibody displaying the most comparable expression levels between the two assays may be used for screening.

The term antibody refers to an immunoglobulin molecule or combinations thereof that specifically binds to or is immunologically reactive with a particular antigen and includes polyclonal, monoclonal, genetically engineered and otherwise modified forms of antibodies, not limited to chimeric antibodies, humanised antibodies, heteroconjugate antibodies (for example bispecific antibodies, diabodies, triabodies, and tetrabodies), single chain Fv antibodies (scFv), or polypeptides that contain at least a portion of immunoglobulin that is sufficient to confer specific antigen binding to the polypeptide. Antibody fragments include proteolytic antibody fragments such as F(ab′)2 fragments, Fab′ fragments, Fab′-SH fragments, Fab fragments, FV, rIgG, recombinant antibody fragments such as sFv fragments, dsFv fragments, bispecific sFv fragments, bispecific dsFv fragments, complementarity determining region (CDR) fragments, camelid antibodies and antibodies produced by cartilaginous and bony fishes and isolated binding domains thereof. A Fab fragment is a monvalent fragment consisting of the VL, VH, CL and CH1 domains; a F(ab′)2 fragment is a bivalent fragment comprising two Fab fragments linked by a disulphide bridge at the hinge region, an Fd fragment consists of the VH and CH1 domains; an FV fragment consists of the VL and VH domains of a single arm of an antibody; and a dAb fragment consists of a VH domain. A single chain antibody (scFv) is an antibody in which a VL and VH region are paired to form a monovalent molecule via a synthetic linker that enables them to be made as a single protein chain. Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen binding sites. A chimeric antibody is an antibody that contains one or more regions from one antibody and one or more regions from one or more other antibodies. An antibody may have one or more binding sites. If there is more than one binding site, the binding sites may be identical to one another or may be different. For instance, a naturally occuring immunoglobulin has two identical binding sites, a single chain antibody or Fab fragment has one binding site, while a bispecific or bifunctional antibody has two different binding sites.

An example of an antibody which specifically binds FKBPL is the FKBPL rabbit polyclonal primary antibody (ProteinTech IL, USA, Cat no. 10060-1-AP). To detect this primary antibody, an anti-rabbit IgG horseradish peroxidase secondary antibody (GE Healthcare, Cat no. NA934V) may be used.

Alternatively, other proteins which are capable of selective binding may also be used for detection.

For example, FKBPL may be detected using aptamers (for example a single stranded nucleic acid molecule (such as, DNA or RNA) that assumes a specific, sequence dependent shape and binds to FKBPL protein with high affinity and specificity), mirror image aptamers (SPIEGELMER™), engineered nonimmunuoglobulin binding proteins, for example nonimmunoglobulin binding proteins based on scaffolds including fibronectin (ADNECTINS™), CTLA-1 (EVIBODIES™), lipocalins (ANTICALINS™), protein A domain (AFFIBODIES™) or the like.

### Quantification of Gene Expression

Expression level of the FKBPL gene can also be detected. For example, the expression level of the FKBPL gene in placental tissue.

Gene expression levels may be determined using any technique known in the art, for example methods based on hybridisation of polynucleotides (mRNA transcripts), methods based on sequencing polynucleotides or amplifying polynucleotides.

Quantification of mRNA gene transcript in a sample may be performed using, without limitation, northern blotting, in situ hybridisation, RNAse protease assays, PCR based methods such as reverse transcription polymerase chain reaction (RT-PCR) and real time quantitative PCT qRT-PCR. Alternatively, antibodies with binding specificity to nucleic acid duplexes may be used to determine mRNA levels. Microarray techniques using specific binding members for RNAs of interest, for example cDNA or oligonucleotide probes specific for RNAs of interest or antibodies specific for mRNA of interest wherein the specific binding members are plated or arrayed on a substrate, for example a glass slide or a microchip substrate can be used. The specific binding members may be provided on the substrate at an addressable location and the number of addressable locations can vary from, for example at least three, at least 10, at least 50, at least 100, at least 1000 or at least 10,000 or more. In embodiments the number of addressable locations can vary from less than 1000, less than 100, less than 50, less than 10, or less than 5. In such examples the sample is contacted with the array and the arrayed specific binding members can form detectable interactions with targets in the sample. The interactions may be detected using suitable labels. Where oligonucleotide probes are utilised, under appropriate conditions the oligonucleotide probes can “hybridise” to a target nucleic acid sequence to form base-paired duplexes with nucleic acid molecules that have a complementary base sequence. Hybridisation conditions resulting in particular degrees of stringency will vary depending on the nature of the hybridisation method and the composition and length of the hybridising nucleic acid sequences.

Stringent hybridisation occurs when a nucleic acid binds a target nucleic acid with minimal background. Typically, to achieve stringent hybridisation, temperatures of around 1° C. to about 20° C., more preferably 5° C. to about 20° C. below the Tm (melting temperature at which half the molecules dissociate from their partner) are used. However, it is further defined by ionic strength and pH of the solution. Suitable hybridisation conditions would be known to those of skill in the art, exemplary hybridisation conditions are:

Very high stringency (detects sequences that share at least 90% identity)—hybridisation 5× SSC at 65° C. for about 16 hours,
High stringency (detects sequences that share at least 80% identity)—hybridisation 5×-6× SSC at 65° C. for 16 hours, and
Low stringency (detects sequences that share at least 50% identity)—hybridisation 6× SSC at room temperature to 55° C. for 20 to 30 minutes.

An example of a highly stringent wash condition is 0.15 M NaCl at 72° C. for about 15 minutes. An example of a stringent wash condition is 0.2× sodium chloride and sodium citrate (SSC) wash at 65° C. for 15 minutes (see, Sambrook and Russell, infra, for a description of SSC buffer for example 20× SSC made by dissolving 175.3 g of NaCl and 88.9 g of sodium citrate in 800 ml distilled water. Adjusting pH to pH7.0 with HCl (IM) and adjusting volume to IL with distilled water). Often, a high stringency wash is preceded by a low stringency wash to remove background probe signal. An example of a medium stringency wash for a duplex of, for example, more than 100 nucleotides, is 1× SSC at 45° C. for 15 minutes. An example of a low stringency wash for a duplex of, for example more than 100 nucleotides, is 4-6× SSC at 40° C. for 15 minutes. For short probes (for example about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (for example, >50 nucleotides).

The methodology used in PCR methods, for example RT-PCR, will be well known to those skilled in the art.

Some methods may require the isolation of RNA from a sample. Such isolation techniques are known in the art and may utilise commercially available RNA isolation kits from manufacturers such as Qiagen.

### Specifically Binds

By specific binding is meant a preferential interaction between one binding partner and another binding partner, for example a primer and a target sequence or a protein specific antibody and a protein. Interactions between one binding partner and another binding partner may be mediated by one or more, typically more than one, non-covalent bonds. An exemplary way of characterising specific binding is by a specific binding curve.

### Contacting the Sample

By contact is meant to bring an agent into close proximity with another agent such that both agents can interact with each other. For example an antibody or other binding member may be brought into close proximity with a protein in a sample and where the antibody has binding specificity for the protein, the antibody will bind the protein.

Alternatively, a first nucleic acid may be brought into close proximity with a second complementary nucleic acid (a primer with a target sequence) and can be incubated such that binding may be detected or amplification of the target sequence may occur.

### Biological Sample

A biological sample can be a blood sample, or a plasma or serum sample derived from a blood sample, or cells or tissue isolated from a subject using standard procedures.

For example, a placental sample may be taken from the pregnant woman. This may be done by chorionic villus sampling (CVS). This procedure involves removing a small sample of cells from the placenta. This procedure may be carried out via a transabdominal route where a needle is inserted through the woman's abdomen. Alternatively a transcervical route may be used where a tube or small forceps are inserted through the cervix.

The placental sample may be fixed in formalin and embedded in paraffin (FFPE) or snap frozen using liquid nitrogen.

### Decrease in Expression Level

The FKBPL expression levels from the first time point and the second time point are compared. In women who have an increased risk of PE, the inventors found a statistically significant decrease in FKBPL expression at the second time point taken later in pregnancy, compared with the first time point taken earlier in pregnancy. That is, the FKBPL expression levels decrease as pregnancy progresses.

In more detail and with reference to the examples, when all blood samples were analysed using Wilcoxon signed-rank test, there was an overall significant decrease of FKBPL between time point 1 and time point 2 (p=0.013) when all of the women within the group who developed PE were combined together. There was no difference between the samples taken at these time points within any of the other groups tested.

A decrease in expression level may be at least about 1° A, at least about 2%, at least about 5%, at least about 10%, at least about 20%, at least about 25%, at least about 30%, at least about 50%, at least about 75%, at least about 100% or more.

Alternatively, the expression level may be decreased at least about 2 fold, at least about 3 fold, at least about 5 fold, at least about 8 fold, at least about 10 fold, at least about 20 fold, at least about 100 fold.

For example, FKBPL expression level can decrease in plasma from 3 ng/ml at time point 1 to 1.5 ng/ml at time point 2.

A decrease can also be seen compared with healthy women. That is, women who do not go on to develop PE. For example, in the non-diabetic cohort of women, the interim analysis described in detail in the Experimental section below shows that FKBPL concentration in plasma at 15 weeks gestation was significantly (p<0.01) lower (0.8819 ng/ml±0.03893 SEM) in women who developed PE than in healthy controls (1.036 ng/ml±0.04312 SEM). Samples were matched for age and BMI.

Where protein expression is determined by immunohistochemistry, the expression level may be measured semi-quantitatively, for example with protein expression levels being noted 0, −1, −2 and −3 with 0 being no detectable decrease in expression from control and −3 being the highest detected decrease in protein expression level.

A statistical computer package may be used, for example IBM SPSS Software, to analyse the data. A pooled analysis of samples will have approximately 90% power to detect a medium Cohen's effect size of 0.5 (i.e. a difference in means whose magnitude is half a standard deviation) as significant at the 5% significance level. Independent sample t-tests can be used to provide comparisons between the groups tested and these will be supported by analyses of covariance to adjust for possible confounders. Distributions will be divided using tertiles to permit the relationship between the biomarker levels and PE risk to be examined for threshold effects or non-linearity using logistic regression.

### Increased Risk

By “increased risk”, in the context of the present invention this is to reflect a higher relative degree of risk. The present inventors have found that a decrease in FKBPL expression or secretion is a strong and early indicator of the risk of developing PE. In women who have an increased risk of developing PE, there is a statistically significant decrease in FKBPL expression at the second time point taken later in pregnancy, compared with the first time point taken earlier in pregnancy. By increased risk is meant a pregnant woman who is more likely to develop PE compared to a pregnant woman who does not show a decrease in FKBPL expression at the second time point.

### Time Points in Pregnancy

The time points are taken according to the number of weeks of pregnancy. This is calculated based on the last menstrual period. The first day of the last menstrual period is counted as day 1 of the pregnancy. Week 1 of pregnancy begins one week from the first day of the last menstrual period. Week 2 of pregnancy begins 2 weeks from the first day of the last menstrual period.

The inventors have found that a decrease in FKBPL levels from early pregnancy to late pregnancy is indicative of a higher risk of developing PE.

A first sample may be taken at a first time point early in pregnancy. For example, weeks 3-19; or any range of weeks starting from 3-, 4-, 5-, 6-, 7-, 8-, 9-, 10-, 11-, 12-, 13-, 14-, 15-, 16-, 17- or 18- and ending 4, 5, 6, 7, 8, 9, 10, 11, 12, 13, 14, 15, 16, 17, 18 or 19. For example, the first time point may be between weeks 3-14.

A second sample is taken at a second time point later in pregnancy than the first time point. By later in pregnancy it is meant closer to birth. For example, weeks 10-30; or any range of weeks starting from 11-, 12-, 13-, 14-, 15-, 16-, 17-, 18-, 19-, 20-, 21-, 22-, 23-, 24-, 25-, 26-, 27-, 28-, or 29-and ending 12, 13, 14, 15, 16, 17, 18, 19, 20, 21, 22, 23, 24, 25, 26, 27, 28, 29 or 30.

For example, a first sample is taken at a first time point early in pregnancy, e.g. at 12 weeks. A second sample is taken later in pregnancy, for example, at 15-20 weeks. The expression level of the FKBPL protein or gene is then measured in these samples and the FKBPL amounts in the first sample and the second sample are compared.

Also provided is a method of determining the risk of PE where only one sample is taken. For example, the biological sample may be taken at any range of weeks starting from 5-, 6-, 7-, 8-, 9-, 10-, 11-, 12-, 13-, 14-, 15-, 16-, 17- or 18-, 19-, 20-, 21-, 22-, 23-, 24-, 25-, 26-, 27-, 28-, 29- and ending 6, 7, 8, 9, 10, 11, 12, 13, 14, 15, 16, 17, 18, 19, 20, 21, 22, 23, 24, 25, 26, 27, 28, 29 or 30. For example, the biological sample may be taken between weeks 10-20. This sample is compared to a control, rather than a sample from another time point.

Pregnancy can also be measured in trimesters. A pregnancy is divided into trimesters: the first trimester is from week 1 to the end of week 12; the second trimester is from week 13 to the end of week 26; and the third trimester is from week 27 to the end of the pregnancy. The first sample may be taken in the first trimester. The second sample may be taken in the second trimester. When only one sample is taken, this may be in the second trimester.

A sample may also be taken later in pregnancy from week 30 onwards. For example, from any range of weeks starting from 30-, 31-, 32-, 33-, 34-, 35-, 36-, 37-, 38- and 39- and ending 31, 32, 33, 34, 35, 36, 37, 38, 39 or 40. This may be the only sample taken or this may be when the second sample is taken.

### Control Standard or Control Sample

The control standard may be a numerical value determined from study of the average values from women who do not go on to develop PE. For example, as shown in the experimental section the inventors show that in a non-diabetic cohort of women, interim analysis shows that FKBPL concentration in plasma at 15 weeks gestation was significantly (p<0.01) lower (0.8819 ng/ml±0.03893 SEM) in women who developed PE than in healthy controls (1.036 ng/ml±0.04312 SEM).

The control standard will vary depending on the method of FKBPL expression analysis. Such standards can be determined where a laboratory is processing many subject samples using standardised reagents and equipment.

Instead of a numerical control standard, a control sample may be run in parallel with the biological samples in any method of determining FKBPL expression. A control sample may be from a woman who did not develop PE. Or a control sample may be a sample with a pre-determined concentration of FKBPL which represents that of a pregnant woman without PE. An internal control will usually be analysed at the same time as the subject sample.

Control standards and samples may be matched for age and BMI.

### Clinical Characteristics of PE

Clinical characteristics related to PE can be used in conjunction with FKBPL level monitoring to enhance the predictive value of the FKBPL test. The clinical characteristics monitored may be any one or more of the following: blood pressure, BMI, mean uterine artery resistance index, proteinuria, lipid profile or nutrition.

### Pre-Gestational Diabetes

Pre-gestational diabetes refers to women with Type I or Type II diabetes mellitus which existed before conception.

Additional Pre-Natal Monitoring

If there is a high risk of developing PE, the woman and baby may have additional pre-natal monitoring. For example, the blood pressure of the woman may be checked more regularly. Urine samples will be taken regularly to measure protein levels. Blood tests may be performed to check the woman's kidney and liver function. Additional ultrasound scans may be performed to check blood flow through the placenta, measure the growth of the baby, and observe the baby's breathing and movements. Other relevant biomarkers might also be checked for.

### Methods of Preventing PE

If a pregnant woman is determined to be at high risk of developing PE, therapies which target the FKBPL pathway and cause an increase in FKBPL expression (FKBPL agonists) may be prescribed. For example, statins, optionally pravastatin; fibrates optionally fenofibrate; stilbenoids, optionally resveratrol; or metformin.

### Methods of Treating PE

Treatments for PE may include medications to lower blood pressure (also known as antihypertensive agents). Examples of this type of medication include labetalol, nifedipine or methyldopa.

If you have severe PE, corticosteroids may be prescribed, These can temporarily improve liver and platelet function to help prolong the pregnancy. Corticosteroids can also help the baby's lungs develop in preparation for premature birth.

Severe PE may also be treated with an anticovulsant medication, such as magnesium sulphate, to prevent seizure.

All medication referred to in the above sections is intended to include all pharmaceutically acceptable salts, solvates, hydrates, polymorphs, prodrugs, metabolites, steroisomers, and tautomeric isomers.

Therapeutically effective amount Therapeutically effective amount means an amount of a therapy which will elicit a biological or medical response in the pregnant woman.

### Kit

A kit is any manufacture (for example a package or container) comprising at least one reagent, for example, an antibody, primers or another probe, specifically for detecting FKBPL.

The kit may contain a control sample, such as a tissue sample which is known not to express FKBPL. The kit may include instructional material disclosing how to use the kit to detect FKBPL.

Throughout the specification, unless the context demands otherwise, the terms ‘comprise’ or ‘include’, or variations such as ‘comprises’ or ‘comprising’, ‘includes’ or ‘including’ will be understood to imply the method or kit includes a stated integer or group of integers, but not the exclusion of any other integer or group of integers.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness. Reference to cited material or information contained in the text should not be understood as a concession that the material or information was part of the common general knowledge or was known in any country.

### Examples

Aspects and embodiments of the present invention will now be illustrated by way of example with reference to the following experimentation.

### Example 1

### Determining the Expression Levels of FKBPL in Trophoblast and Endothelial Cells

Methods: Women with pre-gestational diabetes have a higher incidence of PE (four-fold increase) than the general population. Therefore, a diabetic cell model was chosen (see (i) below) as well as a PE cell model (see (ii) below, hypoxia of placenta being a key feature of pre-eclampsia) for evaluation.

Trophoblast (HTR8.SV.neo, BeWo) and endothelial cells (HUVEC) were exposed to:
i. diabetic stimuli, in the form of highly oxidised and glycated low density lipoproteins (HOG-LDL, 25 μg/ml)/4HNE (10 μM) or high glucose (10, 20, 30, 40 mM); or
ii. hypoxia (1%) or DMOG (10 μM), before FKBPL/SIRT1 protein and RNA levels were measured.

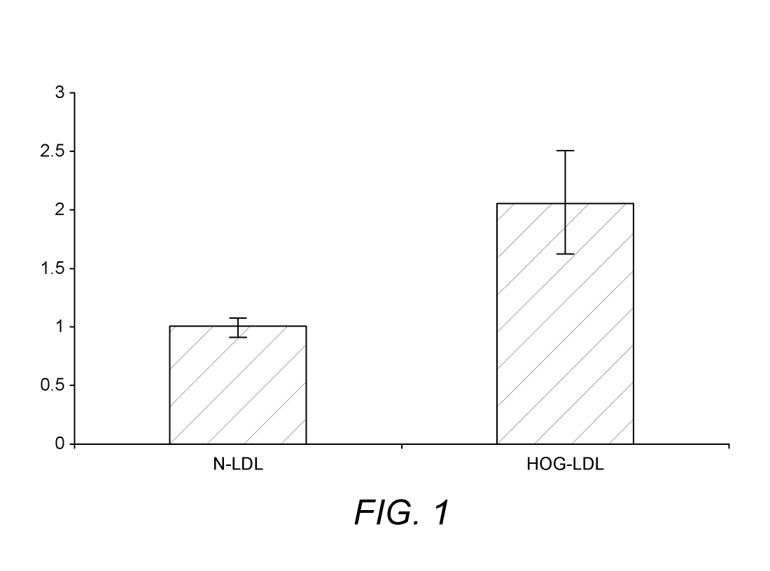

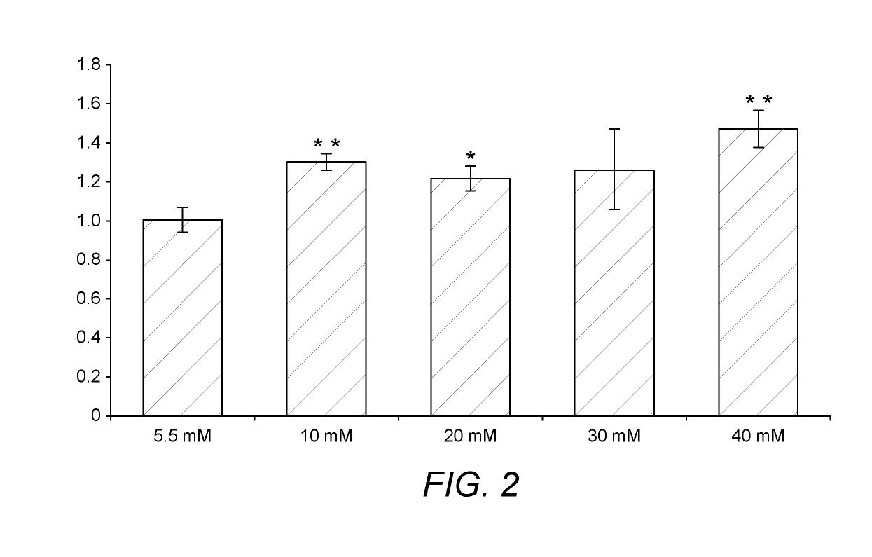

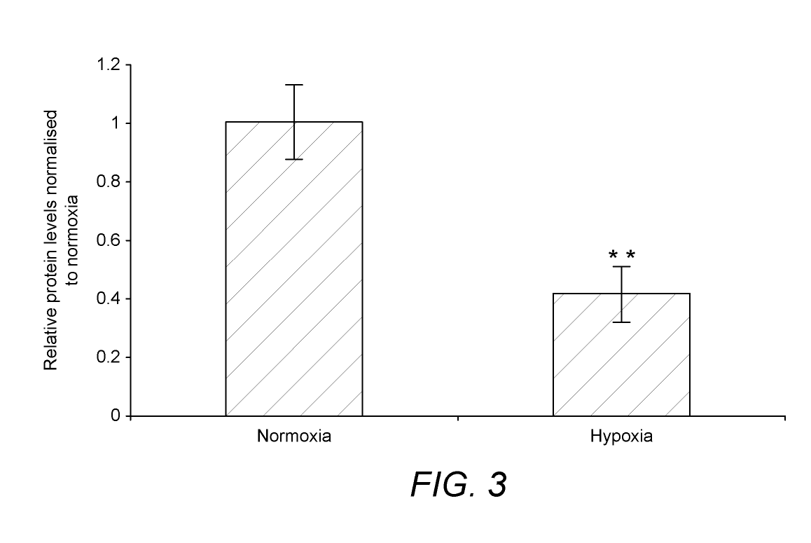

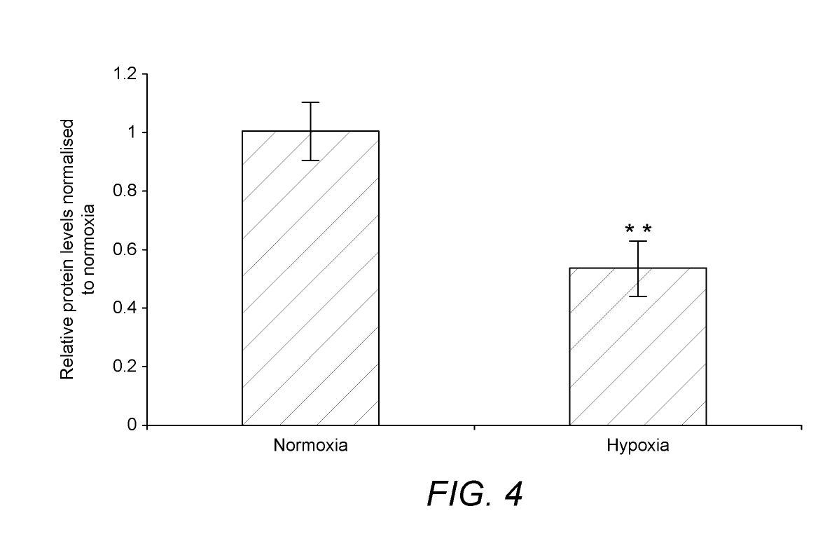

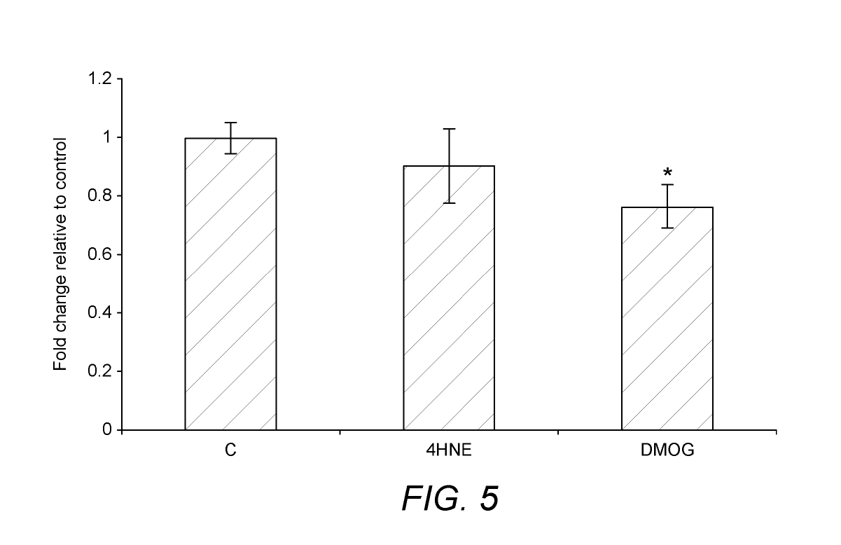

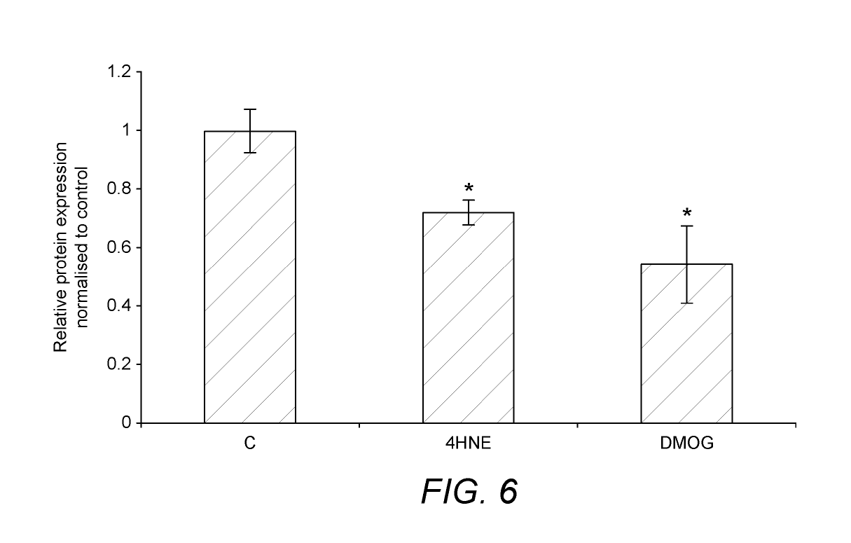

Results: A statistically significant increase in FKBPL protein levels was demonstrated in HTR8.SV.neo cells following treatment with HOG-LDL (p<0.05, FIG. 1). FKBPL mRNA were also upregulated in BeWo cells following 48 h exposure to high glucose environment (10 mM, p<0.01; 20 mM, p<0.05; 40 mM, p<0.01, FIG. 2). Conversely, exposure of HTR8.SV.neo trophoblast or HUVEC/endothelial cells to hypoxia, 1%, for 24 h, led to a downregulation of FKBPL protein expression (HTR8.SV.neo, p<0.01, FIG. 3; HUVEC, p<0.01, FIG. 4). Similarly, treatment with DMOG, HIF-1a activator, led to a reduction in FKBPL mRNA levels in HTR8.SV.neo cells (p<0.05, FIG. 5) and FKBPL protein levels in BeWo cells (p<0.05, FIG. 6). No change in FKBPL mRNA levels was observed when lipid peroxidation product, 4HNE, was used as a treatment in HTR8.SV.neo cells (FIG. 5) in serum containing medium whereas a reduction in FKBPL protein levels was observed in BeWo FKBPL protein levels (FIG. 6). However this was in the presence of the serum whereas treatment with HOG-LDL was in serum-free medium. The inventors hypothesise that serum containing medium may have an anti-oxidant effect which hides the initial increase in FKBPL levels.

### Example 2

### FKBPL is a Predictive Biomarker for PE in Pregnant Women

Expression of FKBPL within placenta was established. The PE biomarker potential of FKBPL was then investigated using patient plasma samples from two prospective longitudinal studies in T1DM and healthy pregnant women.

Methods: Immunofluorescence was performed using FKBPL antibody to establish the expression of FKBPL in chorionic villi of the placental tissue collected upon delivery from a healthy pregnant woman. CD31 protein staining was also performed in order to determine the location of blood vessels/endothelial cells within chorionic

Blood samples were taken from both type 1 diabetic and heathy pregnant women who either developed pre-eclampsia or not. For women with type 1 diabetes blood samples were collected during the first (gestation 12.2±1.9 weeks, [mean±SD]), second (21.6 ±1.5 weeks), and third (31.5±1.7 weeks) trimesters of pregnancy, all before the onset of PE. For women without diabetes, blood samples were collected at 15 weeks gestation. Whole blood was collected into anticoagulant-treated tubes e.g., EDTA-treated (lavender tops) or citrate-treated (light blue tops) or heparinized tubes (green tops). Cells are removed from plasma by centrifugation for 10 minutes at 1,000-2,000× g using a refrigerated centrifuge. Centrifugation for 15 minutes at 2,000× g depletes platelets in the plasma sample. The resulting supernatant is designated plasma. The samples were maintained at 2-8° C. while handling.

A commercial Elisa kit (Cloud-Clone) was used to assay the resulting plasma samples. 100 μl of standards and plasma samples were added to each well and each sample was tested in duplicate.

The samples tested were as follows:

DM-PE− group (Women without diabetes who did not develop PE)
1st trimester—16 samples
2nd trimester—12 samples
3rd trimester—12 samples
   DM+PE− group (Women with T1DM but who did not develop PE)
1st trimester—19 samples
2nd trimester—16 samples
3rd trimester—13 samples
   DM+PE+group (Women with T1DM who did develop PE)
1st trimester—17 samples
2nd trimester—14 samples
3rd trimester—16 samples

In a non-diabetic cohort, plasma samples from healthy women who did not develop pre-eclampsia (n=53) and those who did (n=52) were collected at 15 weeks.

(The sample numbers vary above as it was not possible to obtain all samples for the test)

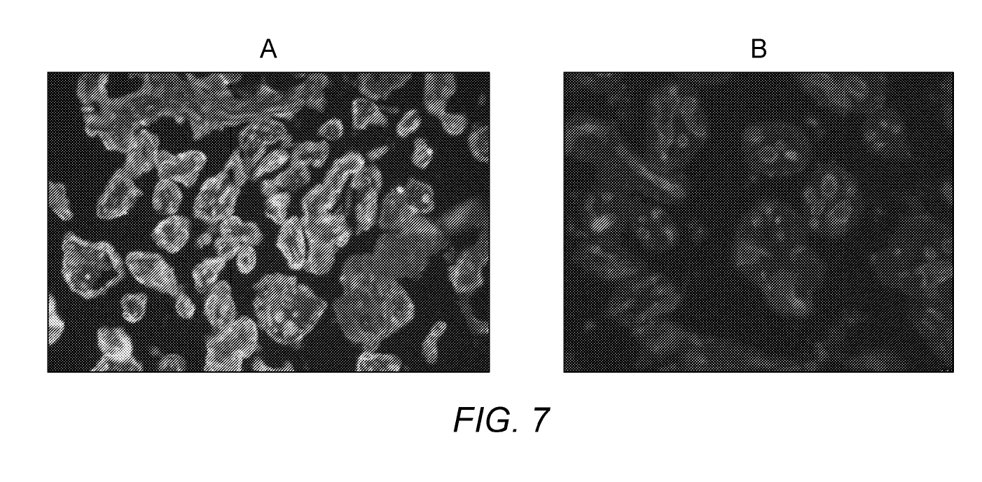

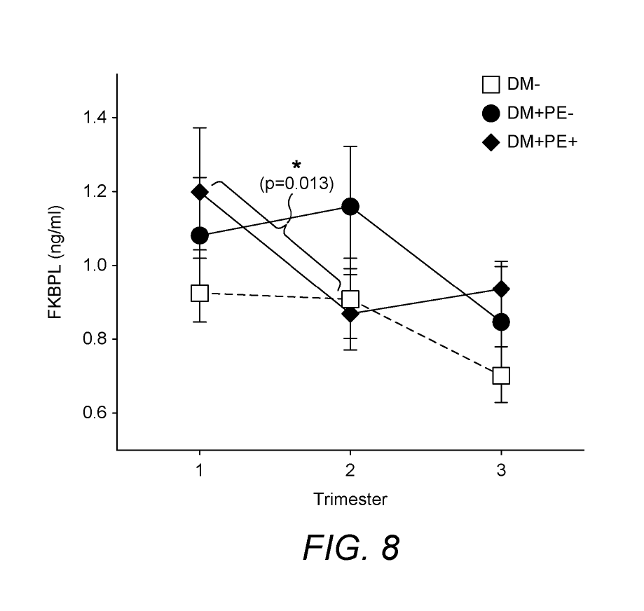

Results: Strong expression of FKBPL was confirmed within placental tissue from healthy pregnancies (FIG. 7, 8). Immunofluorescence staining of placental tissue using FKBPL antibody demonstrated strong expression of FKBPL within syncytiotrophoblasts and blood vessels/endothelial cells (FIG. 8). When secreted FKBPL levels were measured in patient plasma samples, there was a statistically significant reduction in the FKBPL levels between time point 1 (approximately week 12) and time point 2 (approximately week 22) within individual T1DM women who developed PE post 34 weeks of gestation (p=0.013) (DM+PE+). The exact concentration of FKBPL varied between women. However, the results here show a FKBPL concentration range of 0.5-2ng/ml at time point 1, and 0.25-1.5 at time point 2.

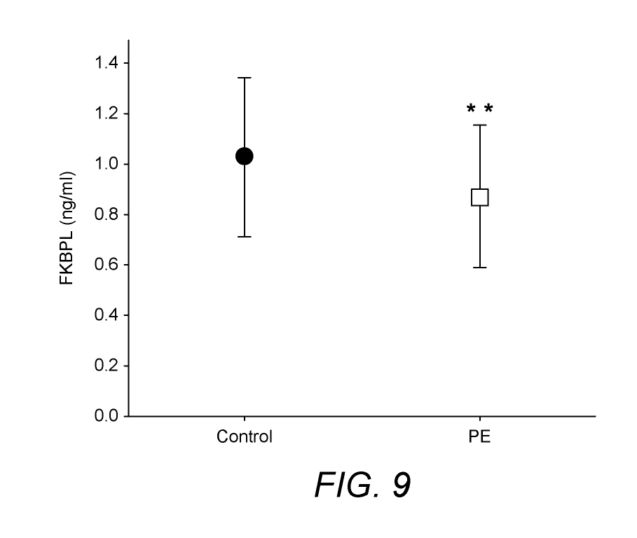

There were no longitudinal changes in the FKBPL levels in T1DM women without PE (DM+PE−) or non-diabetic women without PE (DM−). See FIG. 9. In non-diabetic cohort of women, our interim analysis shows that FKBPL concentration in plasma at 15 weeks gestation was significantly (p<0.01) lower (0.8819 ng/ml±0.03893 SEM) in women who developed PE than in healthy controls (1.036 ng/ml±0.04312 SEM). Samples were matched for age and BMI.

Conclusion: A decrease in the FKBPL protein in plasma was detected early in the second trimester (week 15) in women without diabetes, and towards the end of the second trimester (week 20) compared with visit 1 in type 1 diabetic pregnant women who later went on to develop PE.

Current assay tests (sFlt-1/PIGF) to diagnose PE use biomarkers which are only detectable in the third trimester, around 4 weeks before the onset of symptoms. In contrast, the present test allows an early indication in the second trimester as to the likelihood of the woman developing PE, long before any symptoms appear. The inventors hypothesise that FKBPL may have a role in placental development. Knockdown experiments of FKBPL in mice resulted in embryos which did not survive. Partial FKBPL knockdown in mice produced viable mice embryos however vascular changes were visible in the placenta.

### Example 3

Therapeutic agents which are readily used in patients with diabetes and are safe in pregnancy were investigated for their agonist effect on FKBPL protein expression. These agents were chosen due to their established ability to target associated pathways with FKBPL.

### Methods

Sprague Dawley rats were randomised into non-diabetic (control/sodium citrate injection) or diabetic (Streptozotocin (STZ) induced) groups at 8-12 weeks. A week after injections were administered, both diabetic and control rats were split into two groups, one group was fed Chow with fenofibrate (0.18%) and the second group was fed normal Chow for 5 weeks. Following 5 weeks of treatment with fenofibrate or control, hearts were excised, protein extracted and the protein expression of FKBPL measured using Western blotting.

HTR8.SV.neo cells were treated with fenofibrate or fenofibric acid (10 μM) or vehicle control for 24 h before protein lysates were prepared and FKBPL expression determined using Western blotting.

HUVEC cells were seeded and then exposed to hypoxia (1%) or normoxia (21%) for 24 h in the presence of metformin or vehicle control (VC). Protein lysates were prepared and FKBPL protein level measured using Western blotting.

### Results

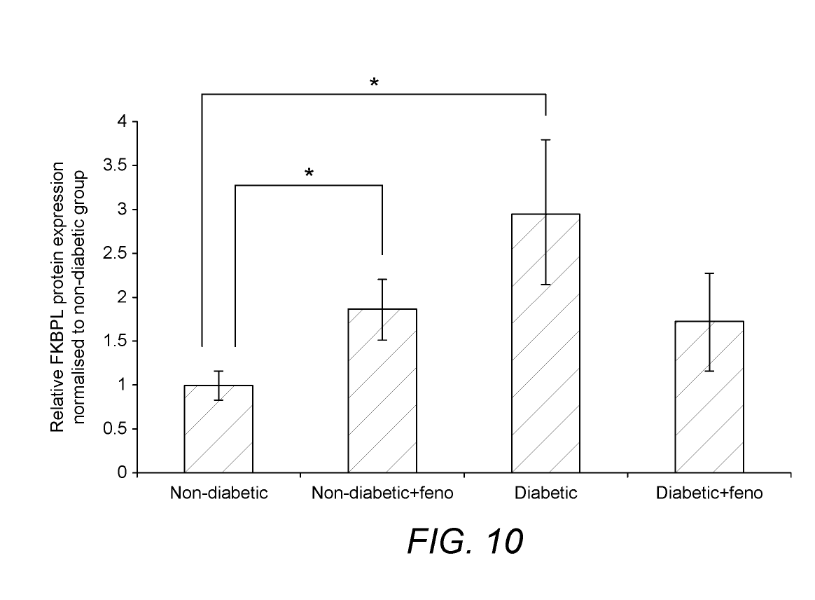

Fenofibrate increases FKBPL protein expression in normal rats without diabetes. Early diabetes time point at 5 weeks also increases the levels of FKBPL (FIG. 10).

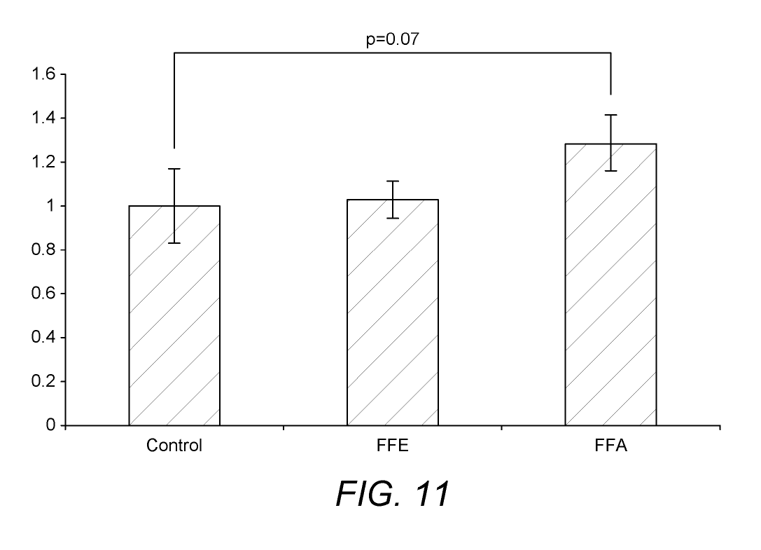

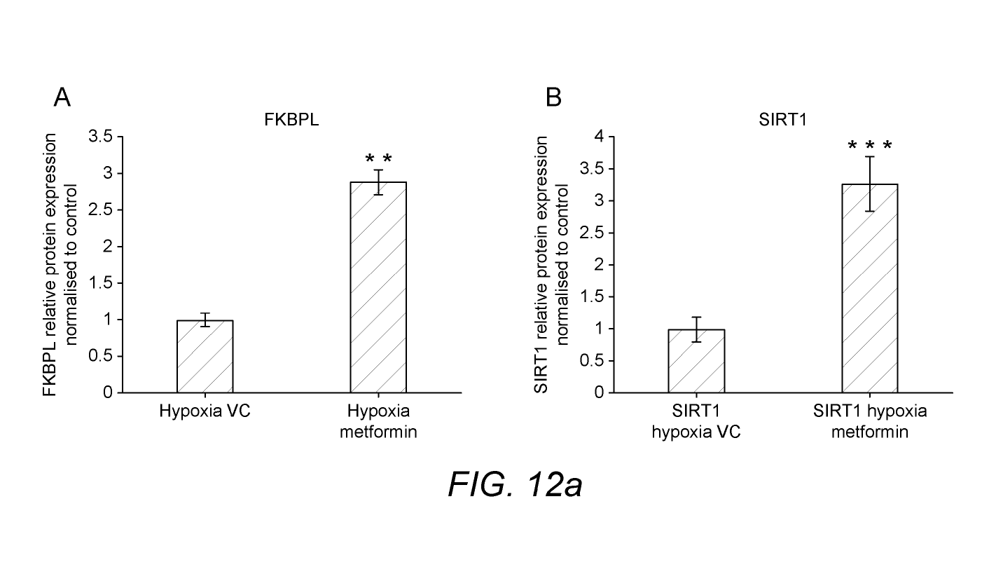

Fenofibrate, an active metabolite of fenofibrate, shows a strong trend towards higher levels of FKBPL compared to control or fenofibrate (FIG. 11) in trophoblast cells.

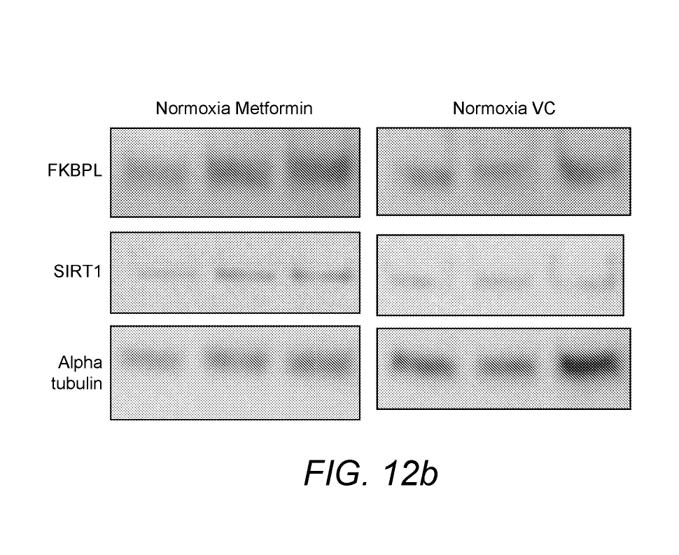

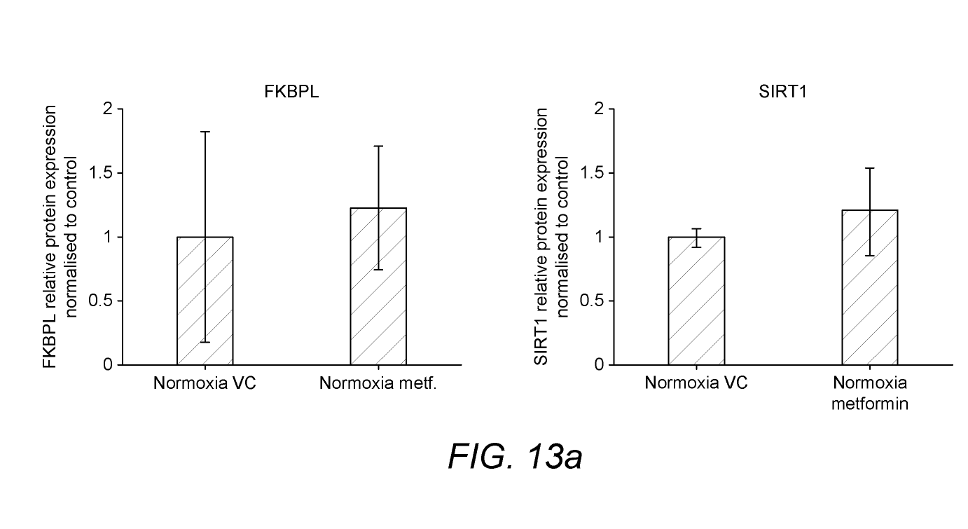

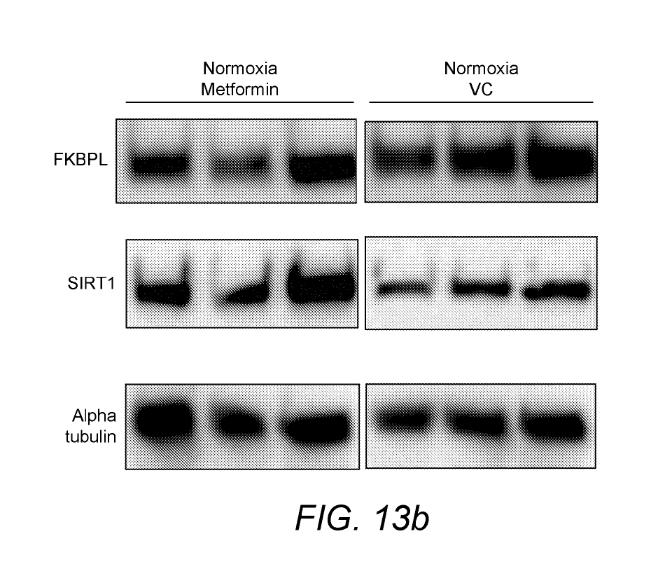

In hypoxia, which downregulates FKBPL, metformin treatment is able to rescue this downregulation by increasing the protein expression of FKBPL (FIG. 12). No change in FKBPL levels was observed in normoxia following treatment with metformin (FIG. 13).

### Conclusion

Fenofibrate or its active metabolite and metformin are capable of acting as FKBPL agonists and therefore could be explored as therapeutic options in pre-eclampsia in conjunction with FKBPL as a biomarker.

1. A method of determining the risk of pre-eclampsia in a pregnant woman, the method comprising:
a) quantifying an expression level of FKBPL in a biological sample taken from the pregnant woman at a first time point;
b) quantifying the expression level of FKBPL in a biological sample taken from the pregnant woman at a second time point, wherein the second time point is later in the pregnancy than the first time point;
c) comparing the expression levels at the first time point and the second time point;
wherein if there is a decrease in the expression level from the first time point to the second time point, the woman is at increased risk of developing pre-eclampsia.

2. (canceled)

3. The method of claim 1, wherein the method further comprises selecting the woman for additional pre-natal monitoring if a decrease in the expression level from the first time point to the second time point is detected.

4. A method of detecting FKBPL in a pregnant woman, the method comprising:
a) taking a first biological sample from the pregnant woman at a first time point; and
b) quantifying the FKBPL in the first biological sample by contacting the sample with an anti-FKBPL antibody and detecting binding between FKBPL and the antibody.

5. The method of claim 4, wherein the method further comprises taking a second biological sample from the pregnant woman at a second time point and quantifying the FKBPL in the second biological sample by contacting the sample with an anti-FKBPL antibody and detecting binding between FKBPL and the antibody, wherein the second time point is later in the pregnancy than the first time point.

6. (canceled)

7. (canceled)

8. The method of claim 4, wherein the method involves first selecting a pregnant woman for testing.

9. The method of claim 5, wherein the FKPBL levels at the first time point and the second time point are compared.

10. A method of treating a pregnant woman at risk for pre-eclampsia, the method comprising:
a) taking a first biological sample from the pregnant woman at a first time point;
b) quantifying an expression level of FKBPL in the first biological sample;
c) taking a second biological sample from the pregnant woman at a second time point, wherein the second time point is later in the pregnancy than the first time point;
d) quantifying the expression level of FKBPL in the second biological sample; and
e) comparing the expression levels at the first time point and the second time point, and
f) administering a therapeutically effective amount of a FKBPL agonist if there is a decrease in the expression level from the first time point to the second time point.

11. (canceled)

12. (canceled)

13. The method of claim 10, wherein the FKBPL agonist is a statin, optionally pravastatin; a fibrate, optionally fenofibrate; a stilbenoid, optionally resveratrol; or metformin.

14. The method of claim 1, wherein the first time point is taken between weeks 3-14 of pregnancy.

15. The method of claim 1, wherein the first time point is taken between weeks 10-14 of pregnancy.

16. The method of claim 1, wherein the second time point is taken between weeks 15-30 of pregnancy.

17. The method of claim 1, wherein the second time point is taken between weeks 15-23 of pregnancy.

18. (canceled)

19. The method of claims claim 1, wherein the biological sample is plasma or serum.

20. The method of claim 1, wherein the biological sample is placenta.

21. The method of claim 1, wherein the pregnant woman has pre-gestational diabetes mellitus.

22. The method of claim 1, wherein the expression levels are determined using a protein which specifically binds FKBPL protein.

23. The method of claim 22, wherein the protein is an antibody.

24. The method of claim 1, wherein the method additionally comprises assessing clinical characteristics of pre-eclampsia.

25. -28. (canceled)


SEQUENCE LISTING (4 sequences)

SEQ ID NO: 1 - DNA, 1087 bp, Homo sapiens
atggagacgccaccagtcaatacaattggagaaaaggacacctctcagccgcaacaagagtgggaaaagaaccttcgggagaaccttgattcagttattcagattaggcagcagccccgagaccctcctaccgaaacgcttgagctggaagtaagcccagatccagccagccaaattctagagcatactcaaggagctgaaaaactggttgctgaacttgaaggagactctcataagtctcatggatcaaccagtcagatgccagaggcccttcaagcttctgatctctggtactgccccgatgggagctttgtcaagaagatcgtaatccgtggccatggcttggacaaacccaaactaggctcctgctgccgggtactggctttggggtttcctttcggatcagggccgccagagggctggacagagctaactatgggcgtagggccatggagggaggaaacttggggggagctcatagagaaatgcttggagtccatgtgtcaaggtgaggaagcagagcttcagctgcctgggcactctggacctcctgtcaggctcacactggcatccttcactcaaggccgagactcctgggagctggagactagcgagaaggaagccctggccagggaagaacgtgcaaggggcacagaactatttcgagctgggaaccctgaaggagctgcccgatgctatggacgggctcttcggctgctcctgactttacccccacctggccctccagaacgaactgtccttcatgccaatctggctgcctgtcagttgttgctagggcagcctcagttggcagcccagagctgtgaccgggtgttggagcgggagcctggccatttaaaggccttataccgaaggggggttgcccaggctgcccttgggaacctggaaaaagcaactgctgacctcaagaaggtgctggcgatagatcccaaaaaccgggcagcccaggaggaactggggaaggtggtcattcaggggaagaaccaggatgcagggctggctcagggtctgcgcaagatgtttggctgattaaaagttaaaccttaaaagagaaaaaaaaaaaaaa

SEQ ID NO: 2 - protein, 349 aa, Homo sapiens
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHSGPPVRLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG

SEQ ID NO: 3 - DNA, 1087 bp, Artificial Sequence
  nucleotide sequence of Homo sapiens FKBPL variant
atggagacgccaccagtcaatacaattggagaaaaggacacctctcagccgcaacaagagtgggaaaagaaccttcgggagaaccttgattcagttattcagattaggcagcagccccgagaccctcctaccgaaacgcttgagctggaagtaagcccagatccagccagccaaattctagagcatactcaaggagctgaaaaactggttgctgaacttgaaggagactctcataagtctcatggatcaaccagtcagatgccagaggcccttcaagcttctgatctctggtactgccccgatgggagctttgtcaagaagatcgtaatccgtggccatggcttggacaaacccaaactaggctcctgctgccgggtactggctttggggtttcctttcggatcagggccgccagagggctggacagagctaactatgggcgtagggccatggagggaggaaacttggggggagctcatagagaaatgcttggagtccatgtgtcaaggtgaggaagcagagcttcagctgcctgggcacactggacctcctgtcgggctcacactggcatccttcactcaaggccgagactcctgggagctggagactagcgagaaggaagccctggccagggaagaacgtgcaaggggcacagaactatttcgagctgggaaccctgaaggagctgcccgatgctatggacgggctcttcggctgctcctgactttacccccacctggccctccagaacgaactgtccttcatgccaatctggctgcctgtcagttgttgctagggcagcctcagttggcagcccagagctgtgaccgggtgttggagcgggagcctggccatttaaaggccttataccgaaggggggttgcccaggctgcccttgggaacctggaaaaagcaactgctgacctcaagaaggtgctggcgatagatcccaaaaaccgggcagcccaggaggaactggggaaggtggtcattcaggggaagaaccaggatgcagggctggctcagggtctgcgcaagatgtttggctgattaaaagttaaaccttaaaagagaaaaaaaaaaaaaa

SEQ ID NO: 4 - protein, 349 aa, Artificial Sequence
  amino acid sequence of Homo sapiens FKBPL variant
METPPVNTIGEKDTSQPQQEWEKNLRENLDSVIQIRQQPRDPPTETLELEVSPDPASQILEHTQGAEKLVAELEGDSHKSHGSTSQMPEALQASDLWYCPDGSFVKKIVIRGHGLDKPKLGSCCRVLALGFPFGSGPPEGWTELTMGVGPWREETWGELIEKCLESMCQGEEAELQLPGHTGPPVGLTLASFTQGRDSWELETSEKEALAREERARGTELFRAGNPEGAARCYGRALRLLLTLPPPGPPERTVLHANLAACQLLLGQPQLAAQSCDRVLEREPGHLKALYRRGVAQAALGNLEKATADLKKVLAIDPKNRAAQEELGKVVIQGKNQDAGLAQGLRKMFG